(19) United States
(12) Patent Application Publication
(10) Pub. No.: US 2024/0394225 A1
(43) Pub. Date: Nov. 28, 2024

(54) Automated High Compute Resource and Sample Management System for Bioinformatic Pipelines

(71) Applicant: Dayhoff Technologies Inc., Toronto (CA)

(72) Inventors: Jacob Shultis, Toronto (CA); Roozbeh Ebbadi, Toronto (CA); Inder Saini, Toronto (CA)

(21) Appl. No.: 18/673,702

(22) Filed: May 24, 2024

Related U.S. Application Data
(60) Provisional application No. 63/504,291, filed on May 25, 2023.

(51) Int. Cl. G06F 16/17; G06F 16/16

(52) U.S. Cl. CPC G06F 16/17; G06F 16/168

(57) ABSTRACT

An automated system for analyzing bioinformatic sample data which collects data from a user, provides analysis pipelines to users based on the received data, automatically provisions high performance computing resources optimized for a selected analysis and outputs presentation ready data to the user.

### CROSS-REFERENCE TO RELATED APPLICATIONS

This application claims priority to U.S. Provisional Patent Application No. 63/504,291, filed on May 25, 2023, titled Automated High Compute Resource and Sample Management System for Bioinformatic Pipelines, which is herein incorporated by reference in its entirety.

### BACKGROUND

Bioinformatic analysis is a growing field that, at times, requires substantial amounts of computing power to process large data sets. However, many of the facilities and organizations who are performing research utilizing bioinformatics may not have access to the computing resources necessary to perform the desired analysis in a reasonable time. Although some elastic computing services may allow a research entity to obtain enhanced computing power on demand, these resources may not be configured to optimize for a particular data set. The computing power offered by traditional elastic computing providers may be expensive, may not have optimized processors, may lack commonly desired software tools that are commonly utilized for the type of data being analyzed, may require extensive configuration by skilled IT/IS experts, and may provide no additional supportive functionality for users.

A need exists for a solution that will provide a flexible amount of processing power that is combined with software which optimizes the data analysis step with minimal user involvement.

### SUMMARY

Disclosed herein is a comprehensive sample management system providing an algorithmic method for conducting bioinformatic analyses, offering a platform for users to improve efficiency in academic and commercial environments. In certain embodiments, the platform dynamically provisions via logic and technology prioritization to appropriately allocate high performance compute (HPC) (also called high power computing) resources and configuring the hardware aspects with other software, dependencies, and tools necessary to conduct a variety of analyses, intaking user specified files for executing a specified bioinformatic analysis, performing statistical analysis on the results, and generating publication ready charts, graphs, and other visualizations while providing progress updates in real-time.

The platform optimizes via algorithmic calculations the synergy between hardware (cloud and locally hosted) and digital bioinformatic information efficiently. An additional aspect of certain embodiments of the platform is the unique visualization reporting that is generated for practitioners to reach deterministic conclusions. An additional aspect of certain embodiments of the platform is artificial intelligence technology layered in the algorithms to redundantly optimize analysis pathways of hardware and software combinations for processing to iteratively determine the best mix for data analysis and presentation.

The software may receive input data from multiple sources, including, without limitation, (1) user inputs, for example, user uploaded files, such as sequenced sample data and metadata files, and a user selected analysis pipeline; (2) external database data, for example, pipeline requirements, analysis details, type of high performance compute resource, and other contextual data; and (3) high performance compute resource data, for example analysis progress, hardware specifications, log files, availability status, and other contextual data.

In one embodiment, the platform first processes the user input data, and may provide a pipeline user interface to allow a user to indicate a desired analysis pipeline, then provisions the appropriate high performance compute resource (cloud or locally hosted) for the analysis based on hardware requirements, speed, and efficiency. The high performance compute resource may be preconfigured to automatically generate or acquire all other necessary software and tools to perform the bioinformatic requested analysis. The analysis is conducted by programmatically executing algorithms for computer scripts that contain instructions and commands that utilize the preconfigured software. These computer scripts vary per analysis pipeline. The resulting files from the analysis are then processed by a second set of computer scripts that perform statistical analysis on the result data and generate publication ready charts, graphs, and other visualizations. These visualizations and results are then stored in the cloud for the user to access and download. Upon completion of the tasks, the platform then deprovisions the high performance compute resource making it available for future analyses.

The platform further provides a unified user interface for manipulating sample files and their associated metadata. The platform further provides an organized platform and workflow for completing the bioinformatic analysis. In some embodiments, the analysis pipelines are customizable pipelines and the platform provides a user interface for customizing the pipeline or the modules contained therein. In some embodiments, the high performance compute resource determines whether portions of the analysis may be run in parallel. In some embodiments, the visualization data is provided as a markup language file, a vector image file, or other image file.

In some embodiments, the system is configured to record performance metrics/performance indicators (such as, for example, time elapsed, CPU processing power used, GPU processing power used, memory usage) of the provisioned high performance compute resource when running the and utilize those metrics to inform future provisioning of high performance compute resources in future analysis pipelines.

Additional aspects of the platform include the ability to send users prompts or notifications via email, text message (e.g. SMS), or other communicative methods on completion of sample analysis. The platform may also be configured to provide real-time status updates, for a given analysis. In one embodiment, cost and time estimates may be provided, wherein the cost and time estimates may be informed by the time and cost to run similar samples using the platform. The platform may also provide a user interface for manipulating sample files and their associated metadata. The platform may contain functionality to anonymize data, remove personal identifying information (PII), compress biometric sample, and transmit compressed or uncompressed data to cold storage.

### BRIEF DESCRIPTION OF THE DRAWINGS

FIG. 1 depicts a flowchart for processing sequence files using dynamic HPC resources, according to one embodiment of the disclosed sample management system.

FIG. 2 depicts an infrastructure diagram according to one embodiment of the disclosed sample management system.

FIGS. 3A and 3B depict one example sample processing pipeline including encrypting and archiving procedures.

FIG. 4 depicts a user interface in accordance with one aspect of the disclosed sample management system.

FIG. 5 depicts a flowchart for provisioning a high performance computing resource in accordance with one aspect of the disclosed sample management system.

FIG. 6 depicts a project dashboard interface in accordance with one aspect of the disclosed sample management system.

FIG. 7 depicts a pipeline selection interface in accordance with one aspect of the disclosed sample management system.

FIG. 8 depicts a module selection interface in accordance with one aspect of the disclosed sample management system.

FIG. 9 depicts a sample management interface in accordance with one aspect of the disclosed sample management system.

FIG. 10 depicts an analysis results interface in accordance with one aspect of the disclosed sample management system.

FIG. 11 depicts a design formula interface in accordance with one aspect of the disclosed sample management system.

FIG. 12 depicts a project settings interface in accordance with one aspect of the disclosed sample management system.

FIG. 13 depicts a block diagram of an exemplary computer system formed with a processor that may include execution units to execute instructions in accordance with certain embodiments of the present disclosure.

FIG. 14 depicts a flowchart for operation of certain aspects of the disclosed sample management system.

### DETAILED DESCRIPTION

In the following detailed description, reference is made to the accompanying drawings that form a part hereof, and in which the specific embodiments that may be practiced is shown by way of illustration. These embodiments herein are described in sufficient detail to enable those skilled in the art to practice the embodiments herein and it is to be understood that other changes may be made without departing from the scope of the embodiments herein. The following detailed description is therefore not to be taken in a limiting sense.

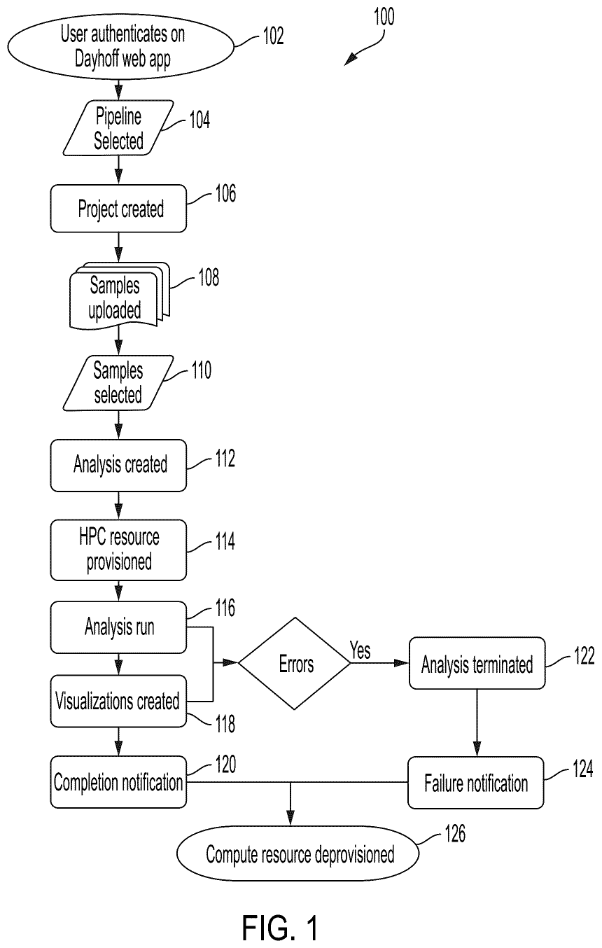

FIG. 1 illustrates a flowchart for the processing of bioinformatic sequence files through the use of dynamic high computing power resources. Bioinformatic sequence files are files which are used to represent bioinformatic or biological data such as DNA, RNA, or other protein sequences. Bioinformatic sequence files may come in a variety of different file formats which may be chosen based on a number of factors including software utilized to create the file, desire to align with industry standards, sample and data type, data storage considerations, or other considerations.

In an exemplary situation a researcher has a set of bioinformatic data that they wish to process. For example, a researcher may commission a lab to perform sequencing on a sample. In an exemplary instance, a researcher may obtain a saliva sample from a human subject, from a rodent, or from some other sample source.

Although a researcher may obtain sequencing from a third-party laboratory, the researcher may also have on-site equipment to perform sequencing. The output of the laboratory or on-site equipment may be a set of data in a particular file format. In order to perform analysis on the sample data, the researcher will require computing resources.

As shown in FIG. 1, to perform the desired analysis a user may need to access the disclosed sample management platform and perform method 100. At step 102, the researcher accesses the platform via a network connection and authenticates via an Internet-accessible application such as a web app, for example via the world wide web or other Internet service. In the embodiment of FIG. 1, the sample management platform is accessible via a web app. The sample management platform offers a user interface that provides a selection of at least one analysis pipeline, each pipeline having a set of scripts to perform analysis of sample data.

The user may then select an analysis pipeline at step 104. A plurality of pipelines may be provided for selection via web application interface. In one embodiment, pipelines are preconfigured by bioinformaticians. The sample management platform may provide details about pipelines, such as a pipeline name, estimated time to run a pipeline, estimated cost, and differentiating factors between selected pipelines.

After a user indicates a selection of a pipeline, a project is created in the online system at step 106, permitting a user to upload sample data via the online web application at step 108. When multiple sets of data are present or uploaded, the user may select a particular data set at step 110.

In some embodiments, bioinformatic pipelines may be customized by a user, i.e., the pipelines may be created or modified via a user interface of the platform. In such embodiments, pipelines are broken up into reusable code modules that are individually responsible for certain tasks such as receiving user-configurable inputs as well as producing unique, user-configurable outputs. For example, user-configurable inputs may include, without limitation, files, metadata, numbers, and strings. Additionally, outputs may include, for example, (without limitation) files, chart data, and images.

In some embodiments, reusable code modules may be preprogrammed options provided by the sample management platform. Reusable code modules may also be customizable modules whose source code may be user-generated or otherwise user-defined. User-generated code modules may be shared among other users of the platform.

The sample management platform may include information regarding the format of input data and output data of each module. When a user is constructing a customized pipeline, the sample management platform may permit a pipeline to include two sequential modules where the output format of a first module aligns with a required input format of a second module. Input/output matching is performed by organizing the inputs and outputs into a data model known as an I/O Rule object. This object can relate to a module as either an input or an output. Each I/O Rule entity has its own unique identifier, as well as a data type. Matching inputs and outputs either occurs if the I/O Rule is the same (same identifier), or if the data type sufficiently overlaps to satisfy the I/O Rule conditions. This helps to validate I/O Rule matching such that a user is prevented from making an error in their analysis by matching two distinct rules that would not provide meaningful input to a specific module.

Modules may include preconfigured tools and software that may be required to run an analysis on high performance computing (HPC) resources. Modules may additionally include input configuration information and output configuration information specific to the module. When a user elects to incorporate a particular module in a customized pipeline, these preconfigured tools, software, and any input/output configuration information may be automatically deployed or provided to a corresponding HPC resource that is provisioned for performing the analysis relevant to the selected module. In some embodiments, HPC resources are provisioned on a per-module basis, however HPC resources may also be provisioned on a per-pipeline basis.

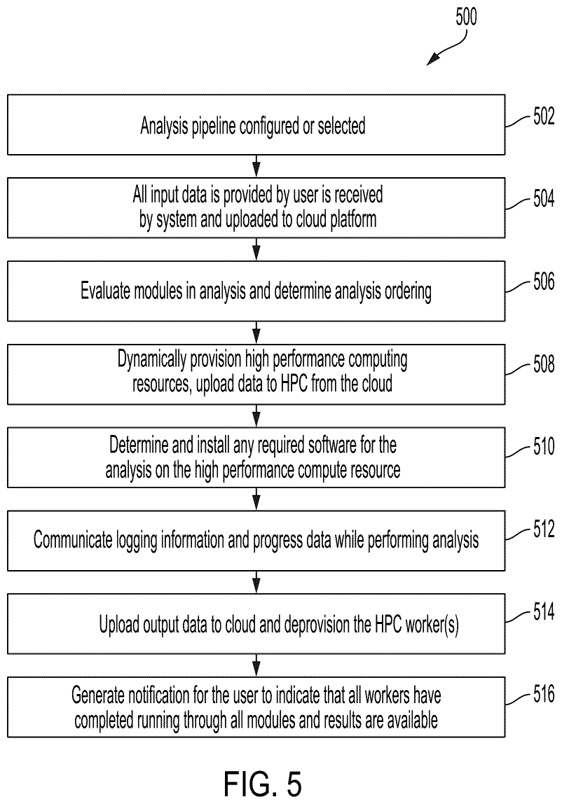

HPC devices may be comprised of a combination of cloud servers as well as locally hosted servers. These servers vary in capability, including GPU enabled hardware, CPU/Memory optimized server instances and high performance servers. These devices are all deployed and operated with the minimum amount of required software to improve performance and reduce storage costs. These devices are typically configured with high memory, large parallel processing capability and sometimes onboard GPU devices for specialized computing tasks. The high performance compute resources differ from traditional general purpose computers by providing significantly higher capabilities in several different aspects of the machine, depending on the analysis being run. The primary capabilities are Random Access Memory (RAM), CPU, GPU and storage capacity. Typically the high performance compute resources will provide substantially more performance in one or more of these capabilities. For example, large datasets may require significantly more RAM to be able to quickly search for gene sequence matches, whereas processing machine learning algorithms may require more GPU performance to optimize matrix computations. In some embodiments, when a particular HPCs or set of HPCs has been provisioned to perform a particular analysis pipeline, the platform is configured to record performance metrics/performance indicators (such as, for example, time elapsed, CPU processing power used, GPU processing power used, memory usage) of the provisioned high performance compute resource when the analysis is performed. The system may make inferences based on that performance data (e.g., comparing run time with provisioned HPC resources to run time for another set of HPC resources, determining that a high percentage of GPU processing power, CPU processing power, or memory is used) and utilize those metrics to derive future provisioning of high performance compute resources in future analysis pipelines. In some embodiments, machine learning and/or AI is used to determine a future set of HPC resources to be used. The provisioning of HPC resources is further described below in connection with FIG. 5.

In one embodiment, researcher may upload data before selecting a pipeline in step 104. When there are multiple sets of data uploaded or present, at step 110 a user may select the samples that the user wishes to utilize for an analysis. The sample management platform may perform pre-analysis of the data to determine a recommended pipeline for use by researcher. Input data may include forward and reverse sequence files (paired-ended), or may contain just a single direction sequence file (single-ended). Upon receipt of the sequence files, method 100 may instruct the platform performing a general integrity check of the received data.

In one embodiment, data input is controlled through a module configuration section of the sample management platform. This step may include displaying a user interface that is inferred by input rules defined for each specific module of an analysis. A user-friendly interface is provided for each input rule to avoid the need for users to interact with module code itself and to provide additional validations on input data. Input data is sanitized and uploaded into the cloud storage system and subsequently to each appropriate module during the analysis.

In addition to providing sequence files, user data may also include metadata about the sequence file or study. For example, such metadata may include a sample identifier, information about whether samples are part of a control group or study group, or other contextual information about the samples (e.g., characteristics of sample subjects). When sample files are provided to the platform, a researcher may decide which metadata to include in the form of custom fields for each sample.

At step 112, an analysis is created. The created analysis defines the configuration used for an analysis run (at step 116). After a user selects a pipeline, the user may be required to provide other configuration options. A user may also be asked to select a reference database. In one embodiment, an analysis is run utilizing multiple reference databases, and results from different reference databases may be compared. In another embodiment, sample management platform may provide user interface options to allow a user to contact a bioinformatician regarding configuration options and pipeline suggestions. Exemplary analysis pipelines include ‘16s’ and ‘Shotgun.’

After the creation of the analysis, at step 114, a high performance computing resource is provisioned. The high compute resource is preconfigured to include all other necessary software and tools to perform the requested analysis. In one embodiment, the sample management platform suggests high computer resources based on the pipeline selected, where the suggestion is based on at least one of hardware requirements, speed, and availability. Suggested hardware may further be based upon other KPIs and prior performing of analysis, e.g., if analysis has been performed on specific pipelines, different performance benchmarks may be established and used to guide selection of a current project. Additionally, sample size and quantity may be utilized in determining provisioning of HPC resources.

The sample management platform may analyze a selected pipeline to determine dependencies between modules. When modules contain interdependencies, method 100 will perform analysis for those interdependent modules serially. When modules do not have interdependencies, or when it is otherwise advantageous, analysis of data may be performed in parallel.

An analysis run is performed at step 116. The analysis is conducted by programmatically executing computer scripts that contain instructions and commands that utilize the preconfigured software. These computer scripts vary per analysis pipeline. Prior to performing a run, the sample management platform verifies that the latest versions of any selected reference database has been obtained. Reference database data may be stored within the sample management and cached for future use. If newer versions of reference databases are available, the platform in method 100 will retrieve the newer data without user intervention. In some embodiments, a researcher may indicate a version of a particular reference database to be used. In some embodiments, multiple selected reference databases are used to allow a user to compare results across multiple reference databases. Benchmark and diagnostic data is stored each time an analysis run is performed so that the platform may utilize that data for later optimization efforts. Additionally, each time a new pipeline is introduced to the sample management platform, the platform creates benchmarking data for future use. Benchmarking data is used to determine what kind of high performance compute resources should be used, including the storage requirements, memory requirements, number of CPUs or GPUs, speed of CPUs and GPUs, preferred cache configurations, or other performance requirements.

After an analysis run is initialized, the system checks or listens for errors. If errors are present, the analysis may be terminated at step 122 and the user and/or an administrator may be notified via email, text message, push alert, phone call, or other notification methodology at step 124. If the analysis run is completed without error, at step 118, the resulting files from the analysis are then processed by a second set of computer scripts that perform statistical analysis on the result data and generate publication ready charts, graphs, and other visualizations. These visualizations are created using software that has been provisioned for the HPC, or utilizing software shared among other platform computing resources. The visualizations and results are then stored in the cloud for the user to access and download via a web interface. During the visualization process, the platform monitors for errors and may send a failure notification at step 124.

Throughout the analysis process the platform may monitor the resources being utilized and optimizes different hardware and software mixes to process the bioinformatic data. For example, if a particular HPC resource or set of HPC resources is useful for a first module of a pipeline, that resource or set of resources may be utilized. However, a subsequent module which uses as its input the output of the first module may benefit from using a different resource or set of resources, and the platform may determine that new resources should be employed in the worker pool carry out the analysis of that subsequent module. This determination may be based on, for example, configuration settings, module information, historical data regarding optimized resources for a module, or other information.

At step 120, a user and/or an administrator may be notified via email, text message, push alert, phone call, or other notification methodology that the analysis run and visualization is complete. Notifications and status updates may also be provided throughout an analysis run. The platform regularly monitors pipeline progress and may report that progress through various means, including via web interface or other application interface.

After notifications have been sent, method 100 deprovisions computer resources at step 126, and made available for future use. One portion of the platform may receive an indication when a particular resource is deprovisioned. Additionally, the sample management platform tracks whether it should have been notified of a deprovisioning of a resource. If such notification has not been received within an expected time, the platform may take additional automated steps to ensure deprovisioning of computer resources. Deprovisioning of resources may prevent waste and cost associated with maintaining resources for an extended period of time or while a resource is not otherwise in use.

The sample management platform may be configured to present pipeline output data to the user. For example, each pipeline module may describe the chart outputs it creates. In some embodiments, pipelines can be configured to generate proprietary publication ready visualizations. In certain embodiments of the sample management platform, charts are completely self-contained markup language files that can be downloaded with their constituent data and viewed independently from the platform. Chart data is sent with its markup language file to the client browser where the underlying data can be manipulated and communicated back to the server to regenerate the markup language file chart file, allowing complete customization of the chart. For advanced pipelines that require statistical analysis, the specific charts that are generated are inferred by the design formula provided by the user during pipeline configuration. The design formula provides the comparisons the user wishes to make between samples based on their metadata. For example, the metadata may include multiple experimental factors, but the user might only be interested in a specific factor, the charts will only be created using the factor of interest. This process determines each permutation of the metadata comparisons based on the user provided design formula and computes and generates all of the charts and their constituent data. All output data may be saved to the cloud so results can be retrieved at any time and can be retained for long periods for archival purposes and to ensure compliance with relevant laws and regulations.

In one embodiment, the data goes through the following steps to generate the final charts: First, statistical analysis is performed on all selected metadata that has been provided as a primary and optionally secondary factor. The statistical data is dispersed to each chart module that converts the statistical data into chart specific output data, this improves the efficiency of chart loading and rendering on the front end since all of the necessary transformations have already been completed. For static charts, this data is then rasterized into image files using R scripts. These image files are stored in cloud storage and are directly loaded by the front end platform. Charts are organized into sub folders and named based on the metadata comparisons they are visualizing, as well as the chart type. For dynamic charts, the data is stored in its raw format in cloud storage. The dynamic chart is rendered using the JavaScript D3 library. The system front-end client downloads the raw data and loads it into the appropriate D3 chart module. The chart module renders the chart for the user, and provides tools to customize the chart (sizing, positioning, labels, colors). It also allows the user to export the dynamic chart as an image file from the client using the D3 library to vectorize to an svg file or raster to a png/jpg image format.

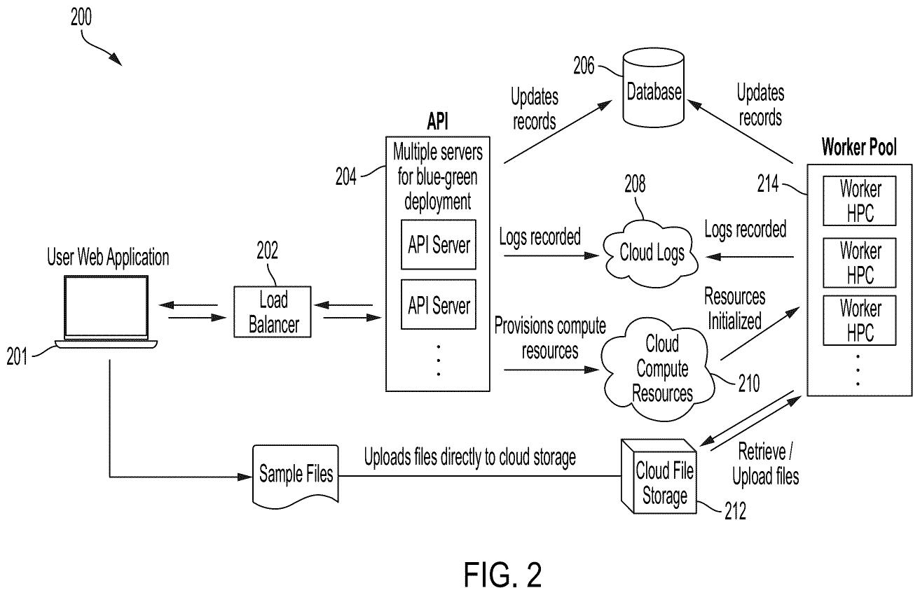

Referring now to FIG. 2, an exemplary infrastructure diagram for sample management system 200 is provided. User web application 201 is depicted and may interact with a load balancer 202 that ensures consistent access to a plurality of API servers 204. API servers 204 coordinate the execution of method 100 for the sample management system 200, permit access to database 206, cloud logs/cloud log storage 208, and cloud compute resources 210, and may also provide access to different networked or cloud-based resources. API servers 204 (as well as cloud compute resources 210) may house substantial operating logic for sample management system 200, and may contain storage and processing to handle interfacing with users as well as perform other processing tasks related to the operation of sample management system 200. API servers 204 may utilize multiple servers to achieve blue-green deployment.

Worker pool 214 comprises a set of high performance computing resources (worker HPCs) that may be initialized by a provisioning request from API servers 204. Worker pool 214 is in communication with database 206 and may update records at database 206, including various forms of information such as benchmarking data, pipeline date, project metadata, or other kinds of data that relate to information regarding pipelines, samples, analyses, or results. Worker pool 214 is further in communication with cloud file storage 212. Worker pool 214 is further in communication with cloud log storage 208, and may input data to cloud log storage site.

API servers 204 may communicate with database 206 to update various records, including pipeline information, sample information, user preferences, project information, or any other information that worker pool 214 may rely upon.

From user web application 201, a user may upload sample files to cloud file storage 212. Cloud file storage 212 may in turn serve sample files to worker pool 214. Worker pool 214 may access sample files that have been stored at cloud file storage 212. Worker pool 214 may also store results, visualizations, and other project data at cloud file storage 212. In one embodiment, when a particular worker HPC (or set of worker HPCs) has completed its analysis, prior to being deprovisioned, a worker HPC will send all its result data to cloud file storage 212, and any updates to cloud log storage 208 or database 206 will be made prior to the worker HPC deprovisioning.

The infrastructure visualization provided is exemplary in nature. Other elements may be added, removed, combined, or used in other ways which will be apparent to one of skill in the art.

In some embodiments, user web application 201 supports an automated archiving system coordinated by API servers 204 that automatically migrates sample files and results data to on-premises or cloud cold storage to reduce storage overhead costs. Users may also define custom archival protocols to customize the logic with which files are moved between cold archival storage and standard rapidly accessible storage.

In certain embodiments, sample metadata is segregated from sample files. Segregation of sample metadata from sample files improves platform performance and search capabilities by housing all of the sample file metadata in a relational platform database. The database acts as a cache so that searching and file management can occur through the API without having to query and manipulate sample files directly.

Sample management system 200 may include several data privacy and security features. For example, sample files and metadata have the option to be encrypted upon ingestion into the platform. Patient metadata has the option to be anonymized through hashing in the platform so that patient information is not exposed to any stakeholders using the platform and would not reveal any sensitive patient data in the event of a breach. Additionally, segmentation of metadata and sample files allows for encryption on either the metadata or sample files so users can effectively choose between efficiency and security for their stored data.

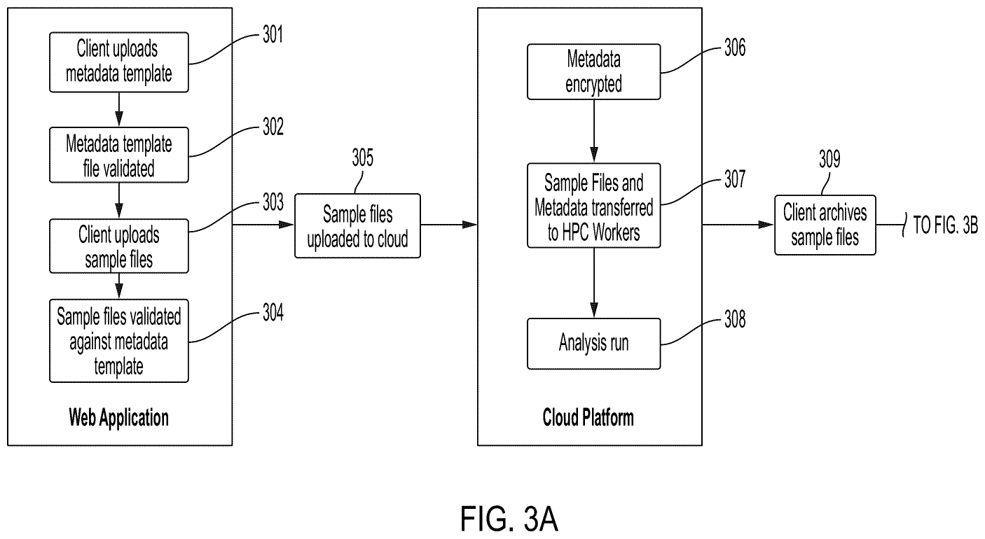

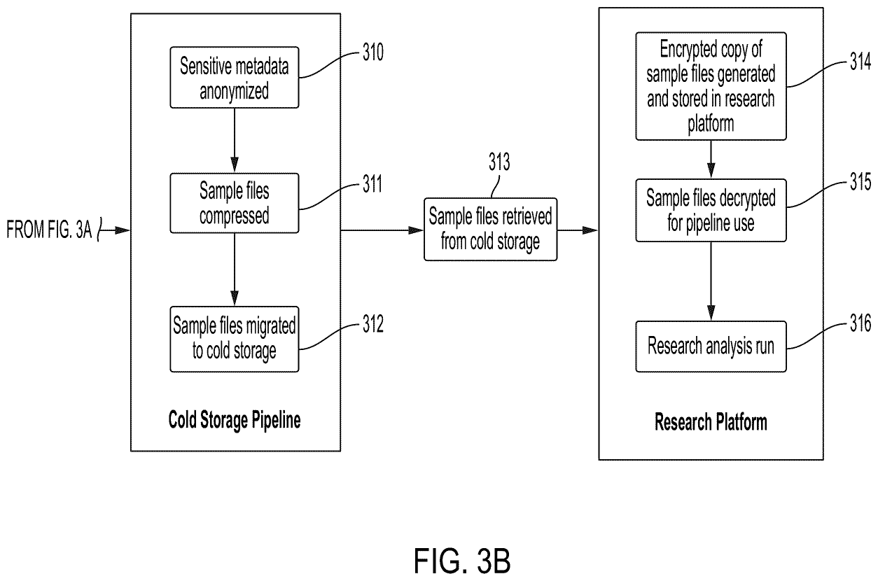

FIG. 3 (in FIGS. 3A and 3B) depicts an exemplary sample processing pipeline including encrypting and archiving procedures. At step 301, a researcher using a client device will upload a metadata template. At step 302, metadata template file may be validated to ensure it is formatted appropriately and may ensure it is free of errors. At step 303, the researcher will upload sample files from a client device. At step 304, the sample files are validated against the previously determined metadata template. Steps 301-304 are typically conducted via a web application/web interface. At step 305, sample files are uploaded to the cloud.

In one embodiment, a cloud platform such as a custom set of API servers or logic deployed on an AWS instance conducts steps 306-308. At step 306, received metadata is encrypted, for example, to ensure subject privacy. At step 307, sample files and metadata are transferred to HPC workers that will be used in analyzing the data. At step 308, analysis runs are performed by the cloud platform. The output of the analysis (not shown) may be stored and utilized by researchers.

At step 309, a researcher may elect to archive sample files. At step 310, any sensitive metadata, e.g., PII of data subjects, is anonymized. At step 311, sample files are compressed, and at step 312, sample files are migrated to cold storage for potential later retrieval.

At step 313, sample files may be requested from cold storage. After retrieval from cold storage, steps 314-316 may be performed at the research platform. For example, an encrypted copy of sample files is generated and stored in the research platform at step 314. At step 315, sample files are decrypted for platform use. At step 316, analysis runs are performed by the research platform. The output of the analysis (not shown) may be stored and utilized by researchers.

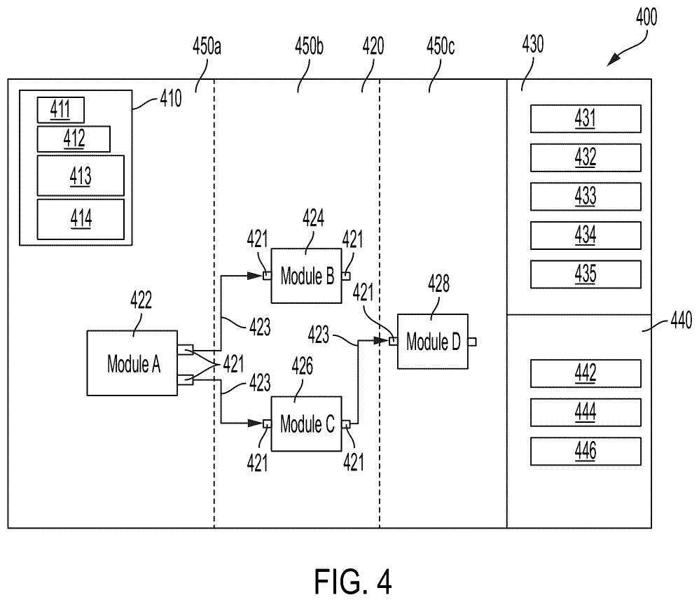

FIG. 4 shows an exemplary user interface for a pipeline building tool in accordance with this disclosure. User interface 400 provides a plurality of user interface elements including rule panel 410, module panel 420, building panel 430, and information panel 440.

Rule panel 410 may present information about various elements within user interface 400. Module panel 420 provides a drag-and-drop interface that visualizes various analysis modules 422, 424, 426, and 428. Module panel 420 shows discrete steps in the analysis pipeline and demonstrates the predicted process flow utilizing arrows and provides information regarding the inputs and outputs from each of the modules. Input/output indicators 421 represent the required inputs and outputs for each module. A name and short descriptor for the elements may be provided via the user interface so that the item may be quickly identified. For example, a user may select a particular input/output indicator 421 and have a pop-up dialog provide specific information about the input/output present, e.g., data format, data type, name, short descriptor, etc. For example, rule panel 410 may present name information in block 411, description information in block 412, or input/output information in blocks 413 and 414. Rule panel 410 may, for example, show the required data type and formatting, as well as any additional validation information, informed by input/output rules provided by the sample management platform. Providing this information to the user assists in preventing data mismatch errors. Rule panel 410 may additionally provide configuration options for the particular input/output indicator selected. In some embodiments, selecting a particular input/output indicator will cause all matching indicators to be highlighted. Alternatively, a user may use an indicating device such as a mouse to hover over the particular output to learn the specific information about the input/output. Alternatively, after selecting a specific input/output indicator 421, specific information may be provided in another portion of the user interface such as information panel 440. Other ways of selecting indicators 421 may be possible, and other presentations of data may be possible. Process flow indicators 423 may be used to show the predicted process flow of the modules. Indicators 423 demonstrate the process order and may show whether modules can be run in parallel or whether modules must be run sequentially.

In one embodiment, module panel 420 may be divided into consecutive horizontal time blocks 450a-450c. Each module which occurs in a particular horizontal time block may be considered for being processed in parallel. Modules which appear in a subsequent time block or previous time block (represented by a horizontal section of module panel 420) must be run serially with respect to one another. For example, module 422 appears in time block 450a. Modules 424 and 426 appear in time block 450b. Module 428 appears in time block 450c. Because module 422 is in a different time block than modules 424 and 426, it must be processed serially prior to modules 424 and 426. Because modules 424 and 426 appear in time block 450b, they may be processed in parallel with one another. In an alternative embodiment, time blocks may be disposed vertically (as opposed to horizontally). In other embodiments, time blocks may be presented using other information such as color-coding. Although three blocks are presented, multiple additional time blocks may be possible.

Building panel 430 presents analysis modules 431, 432, 433, 434 and 435 that may be added to module panel 420 via the drag-and-drop interface. A user electing to include a module, such as module 431 would select that module and place it in module panel 420 at its desired position within the existing analysis pipeline. In one embodiment, building panel 430 presents all modules available the platform. Modules may be searchable by name and may be filtered by capability for quick access.

Information panel 440 lists information about the current analysis as a whole in exemplary fields 442, 444, and 446. Information panel 440 may include analysis statistics such as analysis name fields, predicted time to run the analysis, and a total number of modules, as well as a summary of the required inputs and generated outputs for the entire analysis. In some embodiments, information panel 440 may also provide a listing of intermediate outputs or intermediate required inputs for the entire analysis.

FIG. 5 depicts a flowchart for a process 500 of provisioning high performance compute resources in accordance with one aspect of the sample management platform. At Step 502, an analysis pipeline is configured or selected. At step 504, once the analysis pipeline has been configured, all of the input data provided by the user is received by the system and uploaded to the cloud platform. At step 506, the system will evaluate the modules in the analysis and determine the ordering of the modules using topological sorting, based on the input requirements for each module. In some embodiments, step 506 may also identify if certain modules can be run in parallel. At step 508, the system will then dynamically provision the required high compute resources (the worker or workers) for each module in sequence, uploading the necessary input data from the cloud.

At step 510, the system will install any required software for the analysis onto the high performance compute resource. In one embodiment, this is done automatically by the platform by analyzing the software indicated as required by the modules involved in the analysis. Each module's software requirements are automatically accessed from required code and library repositories and installed one-by-one via the automated process. In one embodiment, this is done as a manual download from the required code and library repositories. In an alternative embodiment this is done all at once by mounting a machine image to the worker server that contains all of the dependencies. In some embodiments, the machine image contains all software required to run an analysis or a specific module, including the operating system to be installed on the HPC. Images with the operating system included may also have removed any extraneous software from the operating system to ensure an optimized deployment.

At step 512, while the worker resource runs its analysis, the worker resource regularly communicates logging information and progress data back to the main platform.

At step 514, once the worker has completed the analysis, it will upload any output data to the cloud and the platform will deprovision the worker. At step 516, once all worker servers have completed running through all modules, a notification will be generated for the user to inform them that the analysis has finished and results are available from the front end client.

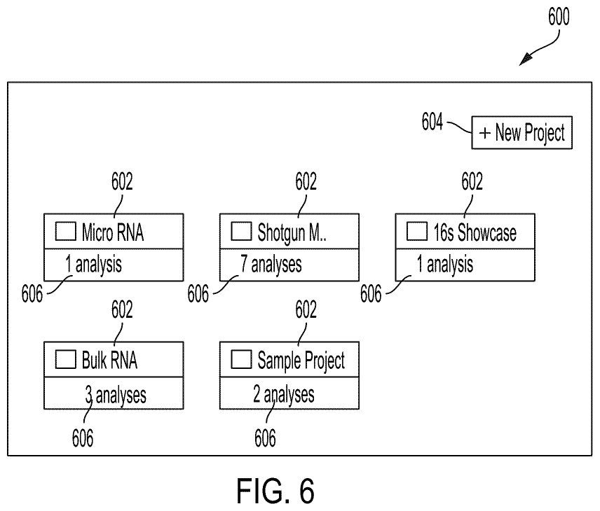

FIG. 6 depicts a portion of a user interface of the system in accordance with one aspect of the system. The system provides a project dashboard 600 which permits a user to select from existing projects 602 or select a user interface element 604 that allows for creation of a new project. Project information element 606 indicates to a user how many analysis pipelines have been created for each particular project.

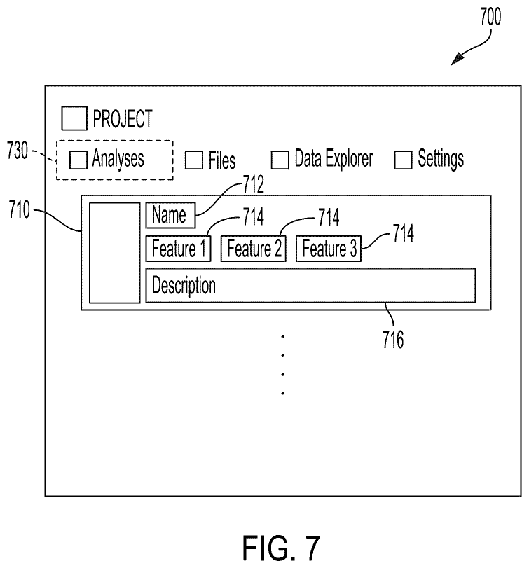

As shown in FIG. 7, when a user selects a project from project dashboard 600, the user is presented with pipeline selection interface 700. Pipeline selection interface 700 presents a plurality of different analysis types that may be selected by a user. Analysis type 710 presents an analysis type that may be selected. Although only a single type is shown, a plurality of analysis types 710 may be presented in pipeline selection interface 700. Name field 712 presents a common name for the analysis type, e.g., 16s, RNAseq pipeline, Shotgun metagenomics pipeline, Micro RNA Sequencing, Multiomics, Single Cell RNA Sequencing, etc. Feature fields 714 provide summary feature information for the given analysis type, e.g., community profiling, whole genome sequencing, small RNA discovery, etc., Description field 716 presents descriptive text to a user regarding the particular analysis. Pipeline selection interface 700 may present multiple pipelines. The pipelines presented may be based on all available pipelines in the sample management system. In another embodiment, the pipelines presented are based upon the data previously loaded for the particular project. The sample management system may use metadata collected from the uploaded data to determine the type of data that is used and may use historical statistics from previous uses of those data types to determine what types of analysis may be useful for a researcher, and thus which pipelines are presented to the user or the order in which the pipelines are presented. Pipeline selection interface 700 may provide a user interface indicator 730 indicating that the active view is the “analyses” view for a particular project.

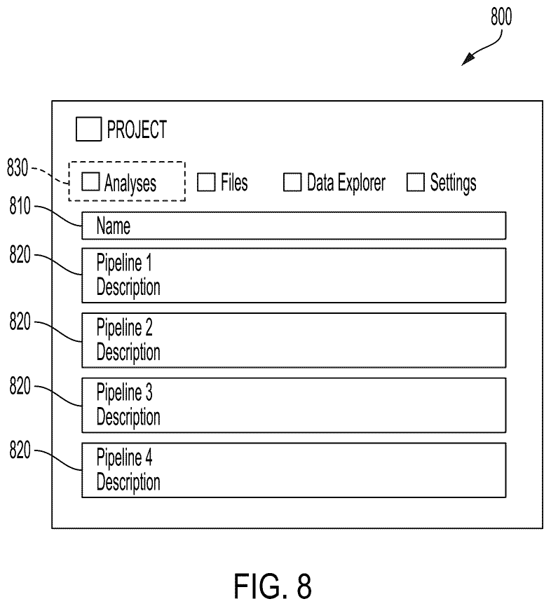

As depicted in FIG. 8, when a user elects to create an analysis within a project, analysis selection interface 800 is presented to the user. Name prompt 810 permits the user to input the name of an analysis. Preexisting pipeline options 820 may be presented to the user to allow the user to selection a particular pipeline, e.g., Shotgun Deep Sequencing, Shotgun Full Analysis, Shotgun Statistical Analysis, and Shotgun Nanopore+AMR. Analysis selection interface 800 may provide a user interface indicator 830 indicating that the active view is the “analyses” view for a particular project.

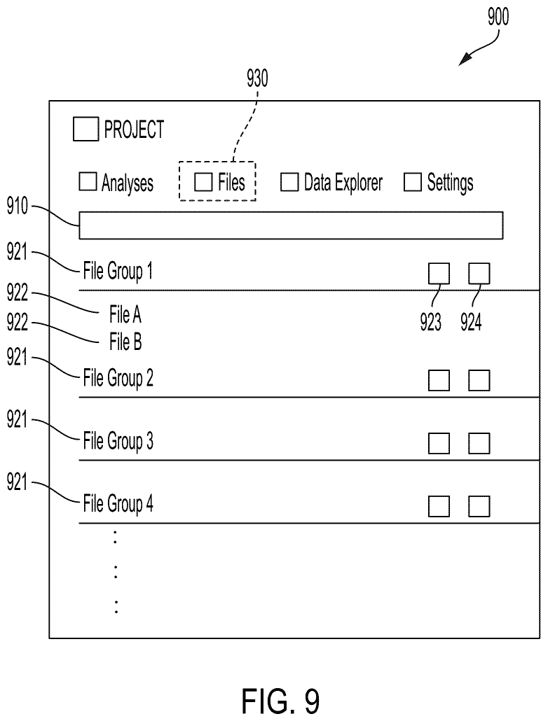

As shown in FIG. 9, the system may present a sample management interface 900. Search field 910 is configured to accept user input to assist a user in locating a single or a set of uploaded files. File groups 921 are presented to the user and may be selected to display files 922 contained within the file groups. File group commands 923 and 924 may be accessed through sample management interface 900 such as deleting or viewing a particular group of files. Files 922 may be listed on sample management interface 900 with additional data such as file size, file name, date created, or other elements. A user interface indicator 930 may indicate that a user is within the sample management interface.

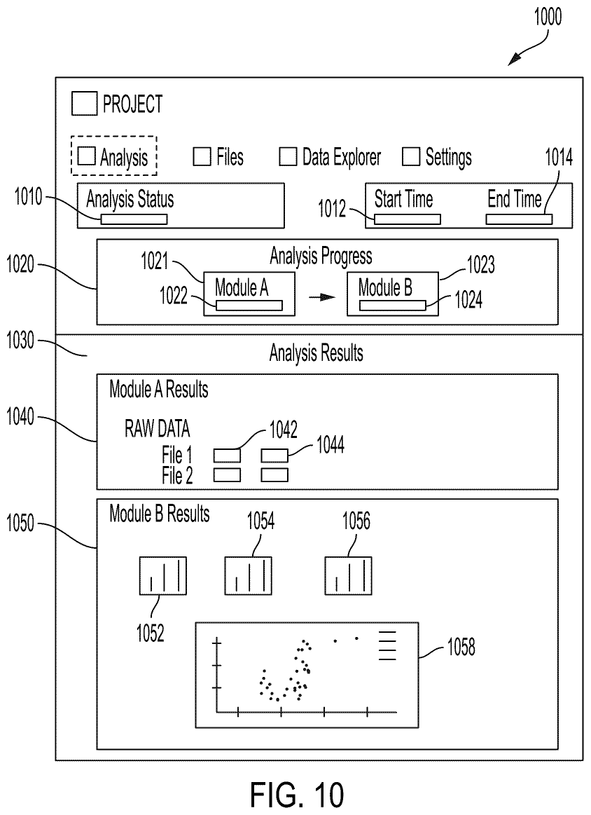

As shown in FIG. 10, analysis results interface 1000 presents certain data results to a user. Analysis status indicator 1010 presents overall analysis status to the user, i.e., complete, not yet started, pending, etc. Start time indicator 1012 and end time indicator 1014 provide information relative to the start and end time of the particular analysis performed. Analysis progress block 1020 provides individual progress indicators for each module in the particular analysis that has been selected. Module indicators 1021 and 1023 identify the particular modules in the analysis, and progress indicators 1022 and 1024 indicate the progress of the analysis as it progresses through these modules. In addition to providing completion data through analysis results interface 1000, the sample management system may be configured to provide alerts to a user via other methods such as, for example, text message, app push notifications, or email. Analysis results section 1030 provides specific analysis information to the user by providing the results for each module. For example, first module results 1040 may include information about a module name, module run time, and may provide access to raw data results. User interface elements such as buttons 1042 and 1044 provide data viewing options to the user, such as the ability to download raw results or view raw results.

Second module results 1050 may provide additional options to the user, including final visualization and chart options. Thumbnail charts 1052, 1054, and 1056 are provided to the user for user selection. Selecting a particular thumbnail chart 1052 may cause the user interface to display an enlarged version of a results chart 1058. Thumbnail charts may be generated based upon user input (i.e., the user requests that certain charts be generated from the analysis). Thumbnail charts may also be automatically generated based on historical usage of the system, based on programming of outputs for the system, may be informed by an AI model which determines a set of likely charts that may be useful, or based on other factors. For example, chart outputs may be determined based on a design formula which is selected by the user and specifies the model used to analyze the data.

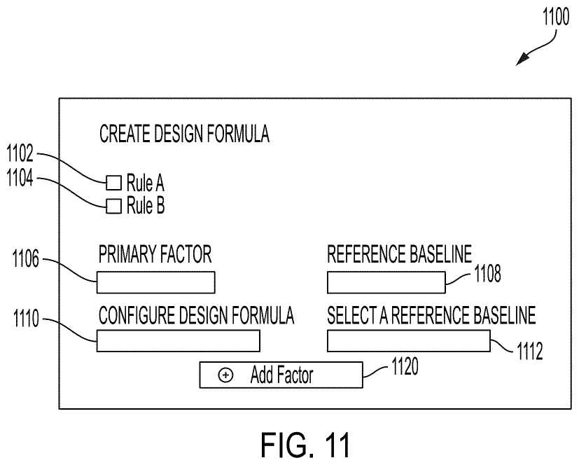

As shown in FIG. 11, sample management system also provides a create design formula interface 1100 for creating design formulae. A design formula describes the relationship between the outcome variable and the experimental conditions in a study and is used to configure the statistical analysis module for pipelines that require it. The design formula is used to generate a design metric, which is used to create the statistical model. In the depicted embodiment, the design formula must conform to two restrictions, rules A and B. Indicators 1102 and 1104 may change to indicate to a user whether the design formula defined in the create design formula interface 1100 conforms to the rules. In other embodiments, different numbers of rules may be provided. User interface elements 1106 and 1108 allow a ruse to select a primary factor and a reference baseline. Factor settings 1110 and 1112 allow a user to configure a particular design formula. Additional factors may be included by utilizing add factor user interface feature 1120.

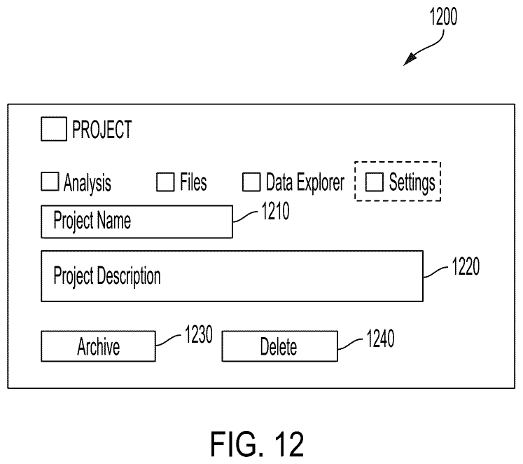

As shown in FIG. 12, sample management system may provide a project settings interface 1200. Interface 1200 may include the ability to rename a project under name field 1210, the ability to provide a project description in descriptor 1220, and the ability to archive or delete a project using user interface archive button 1230 or delete button 1240.

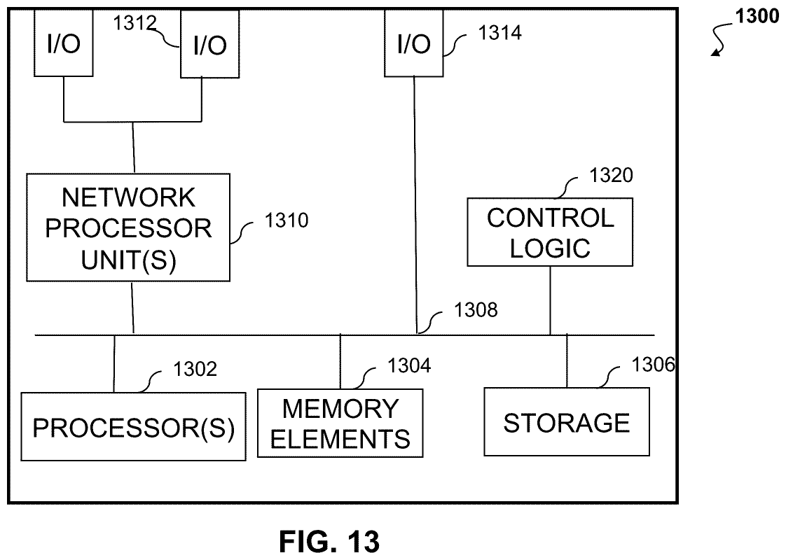

FIG. 13 depicts a block diagram of exemplary computing device which may be used to execute certain portions of the disclosed sample management platform. In at least one embodiment, computing device 1300 may include one or more processor(s) 1302, one or more memory element(s) 1304, storage 1306, a bus 1308, one or more network processor unit(s) 1310 interconnected with one or more network input/output (I/O) interface(s) 1312, one or more I/O interface(s) 1314, and control logic 1320. In various embodiments, instructions associated with logic for computing device 1300 can overlap in any manner and are not limited to the specific allocation of instructions and/or operations described herein.

In at least one embodiment, processor(s) 1302 is/are at least one hardware processor configured to execute various tasks, operations and/or functions for computing device 1300 as described herein according to software and/or instructions configured for computing device. Processor(s) 1302 (e.g., hardware processor(s)) can execute any type of instructions associated with data to achieve the operations detailed herein. In one example, processor(s) 1302 can transform an element or an article (e.g., data, information) from one state or thing to another state or thing. Any of potential processing elements, microprocessors, digital signal processor, baseband signal processor, modem, PHY, controllers, systems, managers, logic, and/or machines described herein can be construed as being encompassed within the broad term ‘processor.’

In at least one embodiment, memory element(s) 1304 and/or storage 1306 is/are configured to store data, information, software, and/or instructions associated with computing device 1300, and/or logic configured for memory element(s) 1304 and/or storage 1306. For example, any logic described herein (e.g., control logic 1320) can, in various embodiments, be stored for computing device 1300 using any combination of memory element(s) 1304 and/or storage 1306. Note that in some embodiments, storage 1306 can be consolidated with memory element(s) 1304 (or vice versa), or can overlap/exist in any other suitable manner.

In at least one embodiment, bus 1308 can be configured as an interface that enables one or more elements of computing device 1300 to communicate in order to exchange information and/or data. Bus 1308 can be implemented with any architecture designed for passing control, data and/or information between processors, memory elements/storage, peripheral devices, and/or any other hardware and/or software components that may be configured for computing device 1300. In at least one embodiment, bus 1308 may be implemented as a fast kernel-hosted interconnect, potentially using shared memory between processes (e.g., logic), which can enable efficient communication paths between the processes.

In various embodiments, network processor unit(s) 1310 may enable communication between computing device 1300 and other systems, entities, etc., via network I/O interface(s) 1312 (wired and/or wireless) to facilitate operations discussed for various embodiments described herein. In various embodiments, network processor unit(s) 1310 can be configured as a combination of hardware and/or software, such as one or more Ethernet driver(s) and/or controller(s) or interface cards, Fibre Channel (e.g., optical) driver(s) and/or controller(s), wireless receivers/transmitters/transceivers, baseband processor(s)/modem(s) and/or other similar network interface driver(s) and/or controller(s) now known or hereafter developed to enable communications between computing device 1300 and other systems, entities, etc. to facilitate operations for various embodiments described herein. In various embodiments, network I/O interface(s) 1312 can be configured as one or more Ethernet port(s), Fibre Channel ports, any other I/O port(s), and/or antenna(s)/antenna array(s) now known or hereafter developed. Thus, the network processor unit(s) 1310 and/or network I/O interface(s) 1312 may include suitable interfaces for receiving, transmitting, and/or otherwise communicating data and/or information in a network environment.

I/O interface(s) 1314 allow for input and output of data and/or information (wired and/or wireless) with other entities that may be connected to computing device 1300. For example, I/O interface(s) 1314 may provide a connection to external devices such as a keyboard, keypad, a touch screen, and/or any other suitable input device now known or hereafter developed. In some instances, external devices can also include portable computer readable (non-transitory) storage media such as database systems, thumb drives, portable optical or magnetic disks, and memory cards. In still some instances, external devices can be a mechanism to display data to a user, such as, for example, a computer monitor, a display screen, or the like.

In various embodiments, control logic 1320 can include instructions that, when executed, cause processor(s) 1302 to perform operations, which can include, but not be limited to, providing overall control operations of computing device; interacting with other entities, systems, etc. described herein; maintaining and/or interacting with stored data, information, parameters, etc. (e.g., memory element(s), storage, data structures, databases, tables, etc.); combinations thereof; and/or the like to facilitate various operations for embodiments described herein.

The programs described herein (e.g., control logic 1320) may be identified based upon application(s) for which they are implemented in a specific embodiment. However, it should be appreciated that any particular program nomenclature herein is used merely for convenience; thus, embodiments herein should not be limited to use(s) solely described in any specific application(s) identified and/or implied by such nomenclature.

In various embodiments, entities as described herein may store data/information in any suitable volatile and/or non-volatile memory item (e.g., magnetic hard disk drive, solid state hard drive, semiconductor storage device, random access memory (RAM), read only memory (ROM), erasable programmable read only memory (EPROM), application specific integrated circuit (ASIC), etc.), software, logic (fixed logic, hardware logic, programmable logic, analog logic, digital logic), hardware, and/or in any other suitable component, device, element, and/or object as may be appropriate. Any of the memory items discussed herein should be construed as being encompassed within the broad term ‘memory element’. Data/information being tracked and/or sent to one or more entities as discussed herein could be provided in any database, table, and register, list, cache, storage, and/or storage structure: all of which can be referenced at any suitable timeframe. Any such storage options may also be included within the broad term ‘memory element’ as used herein.

Note that in certain example implementations, operations as set forth herein may be implemented by logic encoded in one or more tangible media that is capable of storing instructions and/or digital information and may be inclusive of non-transitory tangible media and/or non-transitory computer readable storage media (e.g., embedded logic provided in: an ASIC, digital signal processing (DSP) instructions, software [potentially inclusive of object code and source code], etc.) for execution by one or more processor(s), and/or other similar machine, etc. Generally, memory element(s) 1304 and/or storage 1306 can store data, software, code, instructions (e.g., processor instructions), logic, parameters, combinations thereof, and/or the like used for operations described herein. This includes memory element(s) 1304 and/or storage 1306 being able to store data, software, code, instructions (e.g., processor instructions), logic, parameters, combinations thereof, or the like that are executed to carry out operations in accordance with teachings of the present disclosure.

In some instances, software of the present embodiments may be available via a non-transitory computer useable medium (e.g., magnetic or optical mediums, magneto-optic mediums, CD-ROM, DVD, memory devices, etc.) of a stationary or portable program product apparatus, downloadable file(s), file wrapper(s), object(s), package(s), container(s), and/or the like. In some instances, non-transitory computer readable storage media may also be removable. For example, a removable hard drive may be used for memory/storage in some implementations. Other examples may include optical and magnetic disks, thumb drives, and smart cards that can be inserted and/or otherwise connected to a computing device for transfer onto another computer readable storage medium.

Sample management system may utilize artificial intelligence (AI) and machine learning (ML) to enhance performance and usability. Specific use cases for AI and ML include, for example, the ability to have AI examine the types of samples a user provides and determining the best configuration of an analysis to run, depending on the cell type and quality. As an additional example, a language model may be used to review a statistical analysis and provide summary information to researchers about the statistical analysis. Further, because large sets of data may be presented the user, AI may be utilized in summarizing what data is available, and in highlighting particularly useful information. In another embodiment, and AI model may be trained on results so that a user may ask questions about correlations within the data. In some embodiments, machine learning is used to determine optimal hardware configurations for a particular pipeline.

Machine-learning based classification and training may include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks, or other forms of neural networks. Alternatively or additionally, the classification models can include one or more other forms of machine-learned models such as, as examples, linear classification models; quadratic classification models; regression models (e.g., simple linear regression models, multiple linear regression models, logistic regression models, stepwise regression models, multivariate adaptive regression splines, locally estimated scatterplot smoothing models, etc.); one or more decision tree-based models (e.g., classification and/or regression trees, iterative dichotomiser 3 decision trees, C4.5 decision trees, chi-squared automatic interaction detection decision trees, decision stumps, conditional decision trees, etc.); one or more kernel machines; one or more support vector machines; one or more nearest neighbor models (e.g., k-nearest neighbor classifications models, k-nearest neighbors regression models, etc.); one or more Bayesian (e.g., naïve Bayes models, Gaussian naïve Bayes models, multinomial naïve Bayes models, averaged one-dependence estimators, Bayesian networks, Bayesian belief networks, hidden Markov models, etc.); and/or other forms of models.

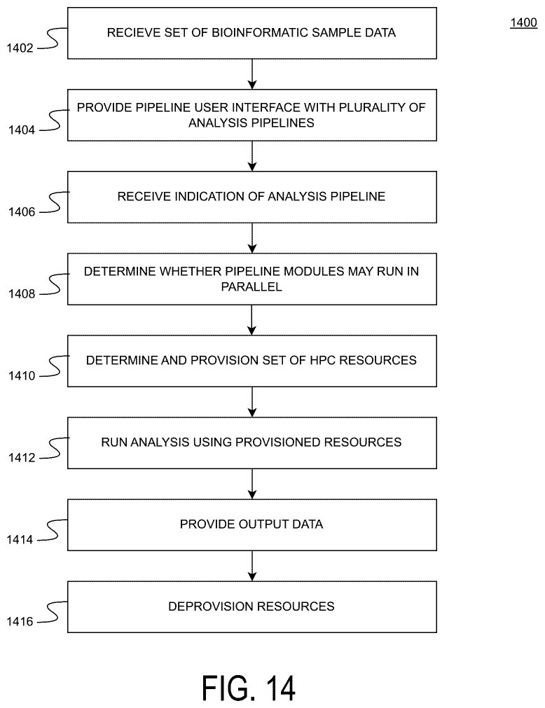

FIG. 14 shows an exemplary method of operation of the disclose sample management system. Method 1400 comprises a first step 1402 of receiving a set of bioinformatic sample data. At step 1404 the system provides a pipeline user interface with a plurality of analysis pipelines for selection by the user. At step 1406, the system receives an indication of a selected analysis pipeline, or may receive indications corresponding to a user constructing its own custom pipeline, by selecting preconstructed modules or building its own modules using scripting or coding. At step 1408, the system determines whether modules within the pipeline may be run in parallel or must be run serially based on the dependencies of the inputs of the modules. At step 1410 the system will determine a set of HPC resources to utilize and provision those resources from a set of available resources in a resource pool. At step 1412 the platform will cause the analysis to be performed on the provisioned HPC resources. At step 1414 output data is collected and provided to a user via a user interface. At step 1416, the provisioned HPC resources are deprovisioned.

It is contemplated that various combinations and/or sub-combinations of the specific features and aspects of the above embodiments may be made and still fall within the scope of the disclosure. Accordingly, it should be understood that various features and aspects of the disclosed embodiments may be combined with or substituted for one another in order to form varying modes of the disclosed disclosure. Further, it is intended that the scope of the present disclosure is herein disclosed by way of examples and should not be limited by the particular disclosed embodiments described above.

We claim:

1. A method for provisioning computing resources for analyzing a bioinformatic pipeline, the method comprising:
receiving a set of bioinformatic sample data;
providing a pipeline user interface displaying a plurality of analysis pipelines;
receiving an indication of a particular analysis pipeline to perform a bioinformatic analysis on the bioinformatic sample data;
determining a set of high performance compute resources to be utilized to process the particular analysis pipeline;
provisioning the set of high performance compute resources;
performing analysis of the particular analysis pipeline using the set of high performance compute resources;
providing output data via an output user interface; and
deprovisioning the set of high performance compute resources.

2. The method of claim 1, wherein the providing a pipeline user interface further comprises providing a customizable analysis pipeline, wherein the customizable analysis pipeline comprises at least one custom analysis module.

3. The method of claim 2, wherein the at least one custom analysis module comprises a set of user-defined instructions.

4. The method of claim 1, wherein the determining the set of high performance compute resources is based on at least one of hardware requirements, speed, and availability relative to the particular analysis pipeline.

5. The method of claim 4, the method further comprising:
evaluating a set of processing steps required to complete performing analysis of the particular analysis pipeline;
determining that a first processing step of the set of processing steps may be performed in parallel with a second processing step of the set of processing steps; and
processing the first processing step and second processing step in parallel.

6. The method of claim 5, wherein the providing output data further comprises generating visualization data, wherein the visualization data provided in a markup language file.

7. The method of claim 6, the method further comprising providing a status information to a user.

8. The method of claim 6, wherein the providing a pipeline user interface displaying a plurality of analysis pipelines further comprises:
evaluating the set of bioinformatic sample data;
determining a plurality of pipelines relevant to the set of bioinformatic sample data; and
including the plurality of pipelines relevant to the set of bioinformatic sample data in the plurality of analysis pipelines.

9. The method of claim 1, the method further comprising:
recording a set of performance indicators associated with the performing analysis of the particular analysis pipeline;
storing the set of performance indicators; and
utilizing information derived from the set of performance indicators to determine a second set of high performance compute resources to be utilized in performing a second analysis pipeline.

10. The method of claim 9, the method further comprising:
anonymizing metadata contained in the bioinformatic sample data;
compressing the bioinformatic sample data for storage; and
transmitting the compressed bioinformatic sample data to cold storage.
